(19) United States
(12) Patent Application Publication
(10) Pub. No.: US 2025/0134096 A1
(43) Pub. Date: May 1, 2025

(54) METHOD AND SYSTEM FOR ASSOCIATING SECONDARY MATERIALS WITH A TISSUE GRAFT AND TISSUE GRAFTS MODIFIED TO INCLUDE SECONDARY MATERIALS

(71) Applicant: CellTherX Corp, Dallas, TX (US)

(72) Inventor: Barry Markman, Frisco, TX (US)

(21) Appl. No.: 18/826,107

(22) Filed: Sep. 5, 2024

Related U.S. Application Data
(63) Continuation-in-part of application No. 18/585,269, filed on Feb. 23, 2024.
(60) Provisional application No. 63/593,177, filed on Oct. 25, 2023.
(60) Provisional application No. 63/597,243, filed on Nov. 8, 2023.

(51) Int. Cl. A01N 1/02

(52) U.S. Cl. CPC A01N 1/126; A01N 1/142

(57) ABSTRACT

Devices and systems are provided for associating secondary materials with tissue grafts, both ex-situ and in-situ. The secondary materials may be medicants, an external cellular matrix factor, a cell, an enzyme, or an element or compound, such as oxygen. In a method, different secondary materials are delivered to a tissue graft via a delivery device at different times, such as at different stages of a healing process.

### RELATED APPLICATION DATA

This application is a continuation-in-part of U.S. application Ser. No. 18/585,269, filed Feb. 23, 2024, which claims priority to both U.S. Provisional Application Ser. No. 63/593,177, filed Oct. 25, 2023, and U.S. Provisional Application Ser. No. 63/597,243, filed Nov. 8, 2023, which prior applications are incorporated herein by reference in their entirety.

### FIELD OF THE INVENTION

The present invention relates to tissue grafts.

### BACKGROUND OF THE INVENTION

Wound dressings and tissue grafts are commonly used to treat areas of injury, such as the skin. However, existing wound dressings and tissue grafts, and the use thereof, suffer from a number of drawbacks. For example, in the current state of the art, wound dressings and biologic tissue grafts are static in nature. In general, they address a specific phase of healing in that their content is not exchangeable.

Healing of a wound or a transplanted organ must undergo several phases to completion. In wound healing, The first two critical phases are the inflammatory and proliferative phases. Each phase requires different medicants toward a successful end result.

For example, during the initial inflammatory phase of wound healing, medicants directed toward cellular elements to remove compromised tissue are required. Other medicants that assist stem cells in the formation of new microvasculature between the tissue graft and host site is beneficial during the inflammatory phase. Lastly, an oxygen rich environment is beneficial to promote faster healing, healing, better incorporation, greater exchange between the modified tissue graft and the host.

In contrast, during the later proliferation phase of wound healing, the recruitment of fibroblasts to enhance a structural matrix and formation of micro-vessels (Angiogenesis), is critical.

A second example pertains to a transplanted organ. Rejection of the transplanted organ occurs in the early stages after surgery. Introduction of medicants relevant to prevent immunological rejection by the host are critical to this early phase. Secondarily, the physiologic functioning of the transplant organ occurs is the next phase.

Current tissue dressings (both synthetic and biologic), can also be categorized as being singular or composite in structure. Singular tissue grafts may be limited in that they contain a primary element, such as a gel (such as a hydrogel or colloids). A composite graft can contain a gel as well as, a fibrous matrix, stems cells, and a antibacterial agent (an Ag/Antibiotic).

Current dermal tissue grafts are categorized as being acellular or decellular. Current placental grafts are categorized by the elements with the graft, i.e. amnion, chorion, umbilical chord, etc.

In each case, the dressing or graft is static in structure, and may not provide critical medicants, external cellular matrix factors, cells, enzymes, and/or other secondary elements or materials, such as oxygen, which have advantages at different phases of the healing process.

Current surgical transference and incorporation of medicants or other materials critical for wound healing also rely on a normal oxygen environment in the host tissue, which often does not exist. A normal oxygen environment in the wound is dependent on multiple patient variables factors. One of the critical variables is arterial inflow and venous outflow in the host wound site. A hypoxic wound environment my limit the exchange, incorporation of elements that assist in healing of the wound.

However, the matrix within the dermal layer in the skin or tissue graft is complex, relatively dense in structure, and tissue grafts contain multiple anatomic structures. Examples of these dermal structures within the matrix, include but not limited to hair follicle germinal centers, pigment stem cells, sebaceous glands, sweat glands, nerve corpuscles micro sensory endings, and cells supporting the regeneration of the matrix.

Thus, various problems exist when attempting to solve the problems associated with existing wound dressings and tissue grafts.

### BRIEF DESCRIPTION OF THE FIGURES

FIGS. 1A-1C illustrate a method of associating secondary materials with a tissue graft in accordance with an embodiment of the invention;

FIGS. 2A and 2B illustrate embodiments of a device for use in associating secondary materials with a tissue graft in accordance with embodiments of the invention;

FIGS. 3A and 3B illustrate other embodiments of a device for use in associating secondary materials with a tissue graft in accordance with the invention;

FIG. 4 illustrates a system for associating secondary materials with a tissue graft in accordance with an embodiment of the invention;

FIG. 5 is a bottom view of a device for associating secondary materials with a tissue graft of the system illustrated in FIG. 4;

FIG. 6 illustrates another embodiment of a device for associating secondary materials with a tissue graft in accordance with the invention;

FIG. 7 is a perspective, exploded view of a tissue graft medicator in accordance with another embodiment of the invention;

FIG. 8 is a side view of the tissue graft medicator illustrated in FIG. 7;

FIG. 9 is a top view of a base portion of the tissue graft medicator illustrated in FIGS. 7 and 8;

FIG. 10 is a top view of a platform of the tissue graft medicator illustrated in FIG. 8; and

FIG. 11 is a top view of a spacer of the tissue graft medicator illustrated in FIG. 8.

### SUMMARY OF THE INVENTION

One aspect of the invention is a tissue graft having associated secondary materials, such as, but not limited to one or more of: a medicant, an external cellular matrix factor, a cell, an enzyme, or element or compound (such as oxygen). Another aspect of the invention comprises a device or system for modifying a tissue graft, including associating one or more secondary materials with a tissue graft, such as a delivery mechanism.

The delivery mechanism may comprise one or more tissue graft medicators and one or more sources of secondary materials. In one embodiment, the delivery mechanism may be part of a delivery system which includes a wound bandage, cover or other treatment elements.

In one embodiment, a tissue graft medicator comprises: a base having a top and a bottom; a plurality of punches extending from the bottom of the base, the punches having a distal cutting end and a passage extending upwardly from the distal cutting end; at least two guide rods extending downwardly from the base; a platform, the platform having a top surface and a plurality of passages extending into the top surface for accepting the at least two guide rods when the base is in two different positions relative to the platform; and at least one spacer for positioning between the base and the platform, the spacer having a top and a bottom, a main passage therethrough from the top to the bottom through which the plurality of punches may be extended towards the platform, and a passage corresponding to each of the guide rods, through which the guide rods extend into engagement with the platform. The punches of the tissue graft medicator may be used to create partial depth cuts in a top and/or bottom surface of the graft and associate secondary materials with the graft, such as by delivery of such materials through the passages of the punches to the areas of the cuts.

Another embodiment of the invention comprises methods of associating secondary materials with a tissue graft. In one embodiment of a method, different secondary materials are associated with a tissue graft at different times, such as during different periods of a healing process.

Further objects, features, and advantages of the present invention over the prior art will become apparent from the detailed description of the drawings which follows, when considered with the attached figures.

### DETAILED DESCRIPTION OF THE INVENTION

Some aspects of the invention comprise devices and systems for medicating tissue grafts, such as by associating secondary materials with tissue grafts, both ex-situ and in-situ. Additional aspects of the invention comprise modified tissue grafts, including those which have secondary materials associated therewith. Yet other aspects of the invention comprise methods of associating secondary materials with tissue grafts and methods of creating modified tissue grafts, including methods of treatment.

The present invention has applicability to tissue grafts. The tissue grafts may comprise cadaveric human tissue, harvested human tissue, or may comprise amniotic or chorionic tissue (including genetically altered human de-cellular amniotic and/or chorionic tissue), or other tissue. In other embodiments, the tissue grafts may comprise cellular non-human tissue, including cellular and acellular processed grafts. The tissue grafts may also comprise synthetic materials. In some embodiments, the tissue grafts may also comprise one or more layers.

In one embodiment, one or more secondary materials may be associated with a tissue graft. The one or more secondary materials may comprise one or more of a gas, liquid, or other similar material (gel) or may comprise materials which are associated with a gas, liquid or gel, including encapsulators acting as a carrier. The one or more secondary materials may comprise, but are not limited to, one or more medicants, external cellular matrix factors, cells, enzymes, oxygen or other elements, compounds or materials. As described below, the secondary materials may be selected for association with the tissue graft for particular purposes and at particular times, such as relating to different phases of a healing process, different conditions, different anatomical areas and different associated host tissue functionality.

In one embodiment of the invention, one or more secondary materials may be associated with a modified tissue graft. Such a graft may be modified in various manners, such as disclosed in U.S. Patent Application Publication 2021/019290 to the inventor herein, which prior application is incorporated by reference in its entirety herein. As disclosed therein, a tissue graft may be modified to include one or more surface features, such as to create one or more voids, openings or the like in one or more surfaces of the tissue graft.

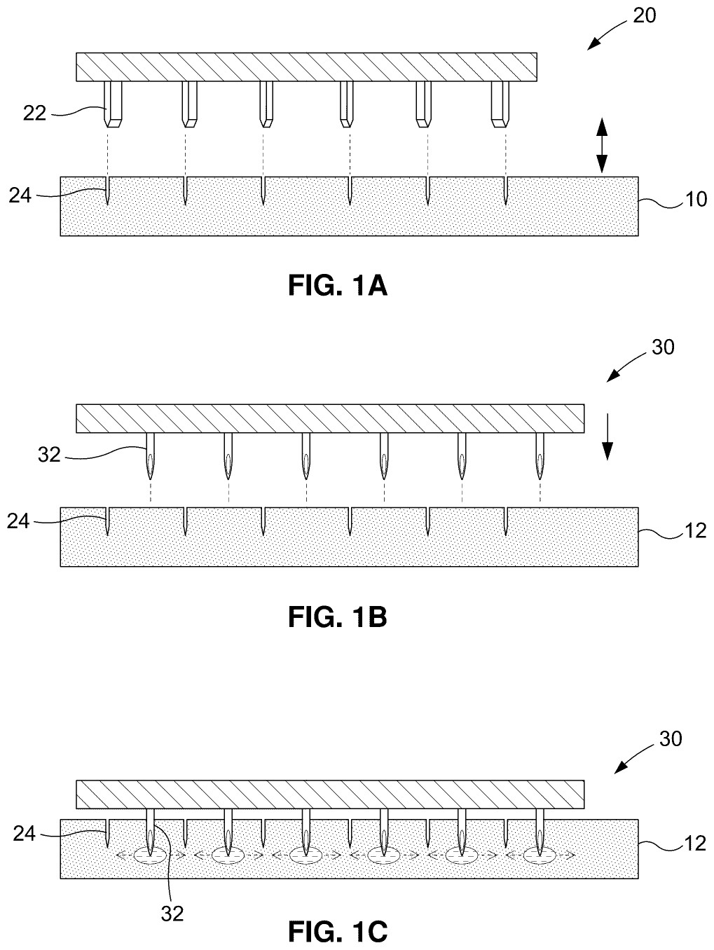

For example, as illustrated in FIG. 1A, a cutting die 20 having a plurality of downwardly extending blades 22 may be pressed into engagement with a tissue graft 10, to create a plurality of cuts 24 (or other openings/voids, etc.), thereby creating a modified tissue graft.

One or more secondary materials might then be associated with the modified tissue graft 12. In one embodiment, secondary materials may be delivered to a tissue graft utilizing a delivery system or delivery device. In other embodiments, the secondary materials may be associated with a tissue graft which is not already modified.

For example, as illustrated in FIG. 1B, a delivery system 30 may comprise one or more delivery members, such as inserters, injectors, blades, cutters or combinations thereof, which may be referred to herein as tissue graft medicators 32, which are used to inject or otherwise associate the one or more secondary materials with (and preferably, within—e.g., into an interior thereof below the exterior surfaces of) the modified tissue graft 12. It will be appreciated that in the preferred embodiment in which the secondary materials are associated with an interior portion of a tissue graft, the tissue graft medicator 32 is preferably configured to pierce, cut or otherwise penetrate the tissue graft. In addition, the tissue graft medicator 32 is also configured to deliver secondary materials, such as via common elements, or in other embodiments, where blades or other cutters are used to cut the tissue graft and other elements, such as needles are used to associate the secondary materials with the tissue graft. Additional details of an example delivery device and system are provided below.

In one embodiment, the delivery system 30 may be used to deliver the secondary materials to regions of the modified tissue graft 12 which have not been modified. For example, relative to a modified tissue graft 12 having a plurality of cuts, the delivery system 30 may be used to deliver the secondary materials to areas of the modified tissue graft 12 that have not been cut (such as between the cuts). This configuration aids in ensuring that the secondary materials do not simply exit the modified tissue graft 12 through the modified area thereof. Instead, as illustrated, the secondary materials may diffuse through the modified tissue graft 12, such as drawn away from the area of injection/introduction and towards the modified areas. This aids in dispersion of the secondary material through the tissue graft. In the illustrated configuration, a first apparatus is used to cut or modify the tissue graft 10 and then a second device is used to deliver the secondary materials to the modified tissue graft 12. In another configuration, however, a single device might include the tissue graft medicator 32, such as having both downwardly extending blades and delivery needles which are spaced from the blades.

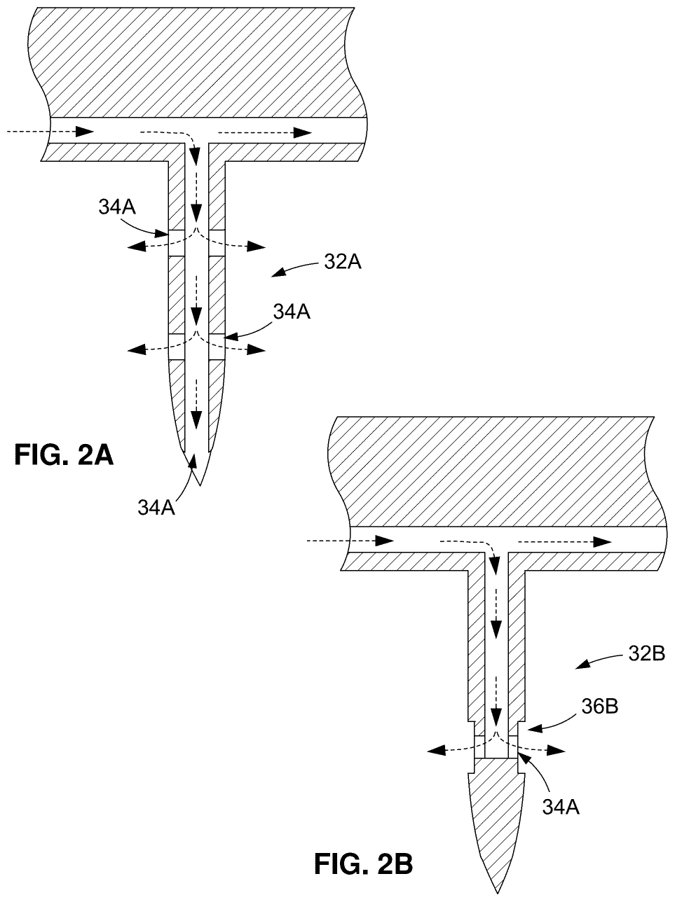

In another embodiment, as illustrated in FIG. 2A, dispersion of the secondary material(s) may be effectuated by delivery through a tissue graft medicator 32A having a plurality of delivery ports 34A, such as in one or more locations in the sidewall thereof, including where the size and/or shape of the ports 134 may vary. It will be appreciated that this embodiment tissue graft medicator 32A might be used to associated secondary materials with a tissue graft 10 or a modified tissue graft 12. In some embodiments, a tip or distal end of the tissue graft medicator 32A might be configured to penetrate the tissue graft 10, while in others, the tip might be configured as a blade or other implement, so as to cut or otherwise modify the tissue graft.

In yet another embodiment, as illustrated in FIG. 2B, a tissue graft medicator 32B may include one or more features for creating a void within the tissue graft. For example, the tissue graft medicator 32B may define an inset 36B, such as in the sidewall thereof, where one or more delivery ports 34B are located. When the tissue graft medicator 32B is inserted into the tissue graft, a void is created due to the inset, creating a space for the infusion or injection of the secondary materials. It will be appreciated that this embodiment tissue graft medicator 32B might be used to associated secondary materials with a tissue graft 10 or a modified tissue graft 12.

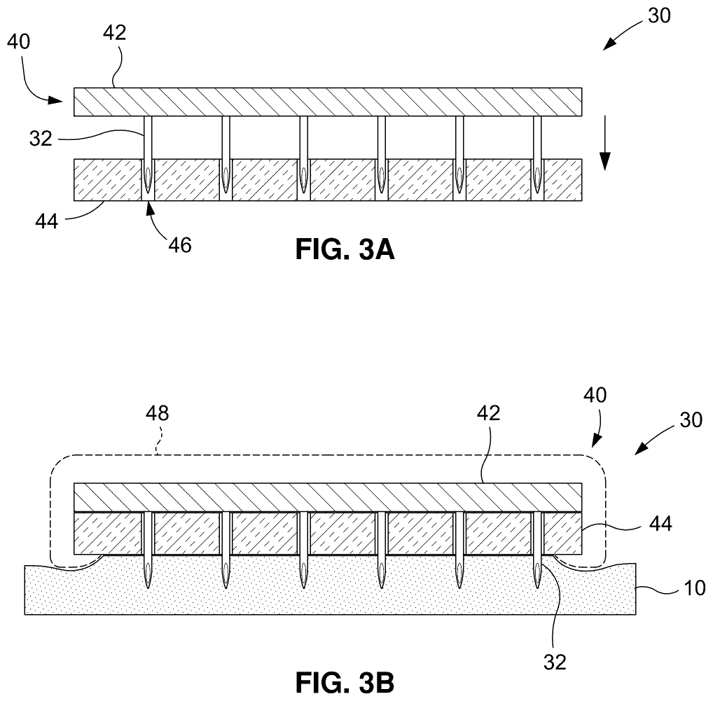

In one embodiment, as illustrated in FIG. 3A, the delivery system 30 may comprise an apparatus or jig 40, such as a mount, to which the tissue graft medicators 32 are mounted and extend. In one embodiment, the tissue graft medicators 32 may be retractable, such as to prevent accidental tissue graft medicator sticks. In one configuration, the tissue graft medicators 32 might be retractable into and out of the jig 40. For example as illustrated in FIG. 3A, the jig 40 might comprise a base 42 to which the tissue graft medicator 32 are mounted, and a guide 44 which defines a plurality of slots or passages 46 through which the tissue graft medicator 32 may be extended (such as from a top side of the guide 44 through to an opposing bottom side thereof), where the base 42 is movable relative to the guide 44. The base 42 may be removable from a first or retracted position in which a distal end of each tissue graft medicator 32 is located in the slots 46, to a second or extended position in which the distal end of each tissue graft medicator 32 extends outwardly of the guide 44, such as for insertion into a tissue graft.

In some embodiments, the base 42 might be biased away from the guide 44 towards the retracted position. This then requires the user to actively press the base 42 towards the guide 44 in order to move the tissue graft medicator to their extended position. In some embodiments, a retaining mechanism or lock might be used to maintain the tissue graft medicator in their extended position, such as when the delivery mechanism 30 is in use.

The base 42 (or the entire jig 40) might be manually moved or it might be moved by a mechanism, such as a motor driven actuator. Such an actuator might include a movement control, such as which controls the range of movement so as to control the depth of insertion of the tissue graft medicator 32. In another embodiment, the jig 40 (or the base 42 relative to the guide 44) might include one or more stops, such as to control the depth of insertion of the tissue graft medicator 32. The stops might be position adjustable, such as to allow the depth of insertion to be varied. For example, different guides 44 might be associated the a particular base 42, where the guides 44 have different thicknesses or depths, thus controlling the distance which the tissue graft medicator 32 extend therefrom.

One aspect of the invention is an off-loading mechanism for a secondary material delivery device. Increased pressure and the effect of shear forces of boney prominences beneath skin and soft tissue is one of the main reasons for the development of pressure injuries. The most common bone prominences that cause pressure injuries include, but are not limited to, the sacrum, hip, and ischial bones. The delivery system 30 may include or incorporate an off-loading mechanism, such as to distribute mass/pressure away from the tissue graft 10.

In some embodiments, the off-loading mechanism may comprise an extension of the delivery system 30, such as at the periphery thereof (such as a peripheral extension of the guide 44), which periphery is located around an interior area which is aligned with the tissue graft 10. In such a configuration, mass/pressure applied to the delivery system 30 may be routed through the peripheral areas to and from tissue surrounding the tissue graft 10, rather than the graft itself.

In another configuration, the off-loading mechanism might comprise or include one or more pads or cushions which are associated with the delivery system 30. For example, as illustrated in FIG. 3B, a cushion 48 might be associated with the delivery system 30, such as the base 42 and/or guide 44. The cushion 48 may comprise a compressible material, such as foam or the like. In other embodiments, the cushion 48 might extend over the sides of the delivery mechanism 30 and/or a portion of the bottom, such as where the delivery mechanism fits into a recess formed in the cushion 48. This embodiment may be beneficial in situations where the delivery mechanism 30 is used in-situ.

As indicated above, one aspect of the invention is a delivery system for delivering secondary materials to a tissue graft, and preferably into a tissue graft, such as via one or more delivery members such as an tissue graft medicator. In one embodiment, the delivery system includes the delivery device and a source of secondary materials, plus a means for delivering the secondary materials to the delivery device.

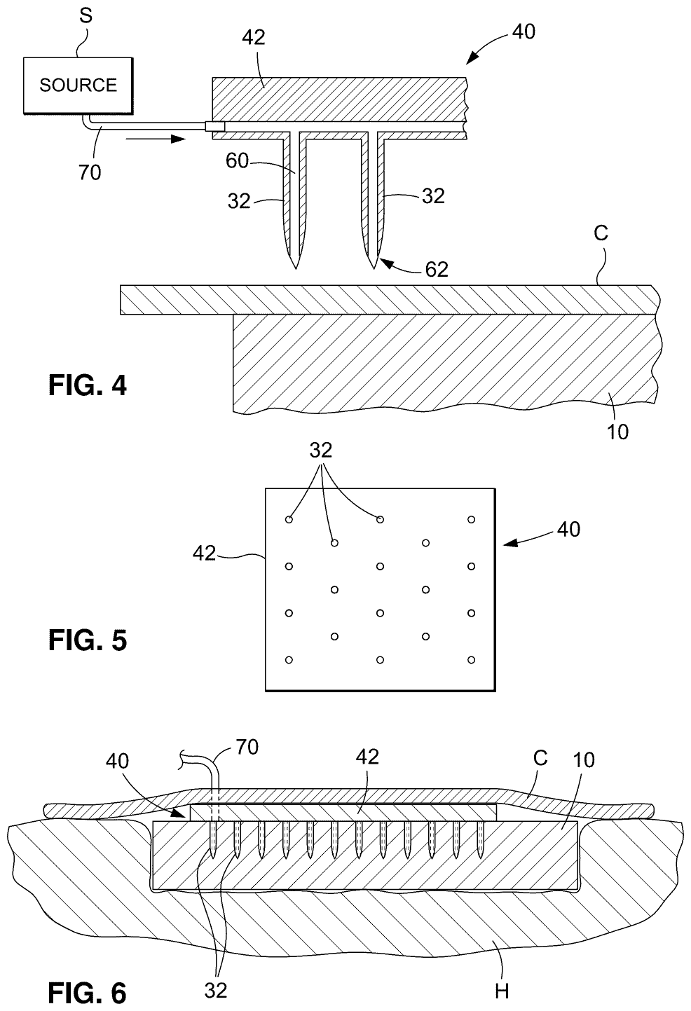

Referring to FIG. 4, in one embodiment, the tissue graft medicator 32 includes at least one delivery passage 60 which extends from at least one secondary material source to the at least one delivery port 62 (as described above, such a port may be located in a sidewall of the tissue graft medicator 32). In one embodiment, the passage 60 may extend from the port 62, such as a through the tissue graft medicator 32, whereby the tissue graft medicator effectively defines an elongated axially extending central passage surrounded by a sidewall.

As indicated above, the tissue graft medicator(s) 32 may be associated with a jig 40 which supports a plurality of the tissue graft medicator 32, such as illustrated in FIG. 5. The number of delivery tissue graft medicator 32, including their arrangement, may vary, such as depending upon the application. In one embodiment, as illustrated in FIG. 5, the proximal end of each tissue graft medicator 32 may be connected to the base 42 of the jig 40, such as where the tissue graft medicator 32 extend outwardly from a bottom side thereof.

In one embodiment, the delivery passage 60 connects to at least one secondary material source S. For example, the source S might be a tank or container of secondary material. At least one inlet or line 70 may lead from the source S to each delivery passage 60, such as through the base 42. In some embodiments, the delivery passages 60 of more than one tissue graft medicator 32 might be connected to a single delivery line, or independent lines might be provided from one or more sources to each tissue graft medicator 32 and associated passage 60. In some embodiments, the lines 70 may be routed (including having shapes) to promote laminar flow of the secondary materials therethrough.

In one embodiment, as illustrated in FIG. 4, one or more secondary materials may be associated with a tissue graft 10 via the tissue graft medicator 32. In particular, the jig 40 and tissue graft 10 are preferably moved relative to one another, such as by lowering the jig 40 downwardly until the distal ends of the tissue graft medicator 32 are located within the tissue graft 10. Secondary materials may then be delivered from the source S to the tissue graft 10 via the one or delivery ports 62 of the tissue graft medicator 32.

In one embodiment, the source S of secondary materials may be pressurized (such as a pressurized tank or container) or a pump may be used to deliver the secondary material from the source S to the tissue graft 10. In one embodiment, the source S might comprise a hospital source, such as an oxygen delivery system in a hospital, or might comprise individual sources, such as containers or tanks. In some embodiments, a single source may be used to deliver secondary materials to a plurality of the tissue graft medicator 32. In other embodiments, a source may be provided relative to each (or several) tissue graft medicator 32. For example, in a portable configuration, the delivery system 30 might enable connection of a source to the jig 40, such as by connecting a cannister of pressurized oxygen to a port defined by or connected to the base 42, which port leads to (such as via one or more lines or passages) the tissue graft medicator 32.

As indicated above, aspects of the invention may be utilized to medicate a tissue graft ex-situ. For example, a tissue graft may be obtained and have secondary materials associated with it before it is placed. As one example, the tissue graft may be obtained, have the secondary materials associated therewith, and then be packaged at one location and then be delivered to another location (such as a hospital) for use, such as application to a patient with a wound.

In another example, as illustrated in FIG. 6, a tissue graft 10 having secondary materials associated therewith might be located under or be associated with another body, such as a secondary tissue layer or a cover C. Such as cover C might comprise, for example, a bandage. The combination bandage with tissue graft may be delivered for use as a package.

In other embodiments, however, the systems and devices of the invention may also be used to deliver secondary materials to a tissue graft in-situ. In one configuration, the delivery system 30 might be associated with or include a wound covering or wound treatment package (such as which may include various other features or elements). In such a configuration, for example, the jig 40 might be located under a cover C thereof. When placed, as illustrated, the tissue graft medicator 32 may extend into the tissue graft 10 as associated with a host H (such as a patient), under a cover C thereof. Secondary material may then be delivered to and associated with the tissue graft 10, including continuously or at one or more times. In some embodiments, a vacuum or suction may be applied, such as to induce circulation of material. In other embodiments, a pressurized environment might be created under the cover C. In such a configuration, the delivery device might be configured as an integrated bandage, wound treatment device or the like which is manufactured and sold as unit and is then associated with the tissue graft once the graft is associated with the host.

Of course, the secondary material delivery device or system might have other configurations and variations. For example, the tissue graft medicator 32 or other delivery elements might be of varied sizes, including lengths (such as to deliver secondary materials at different depths into the tissue graft). The delivery device might be used to deliver different secondary materials, such as the same time (such as from two sources at the same time) or at different times. Further, while the delivery mechanism has been illustrated as being used relative to one side of a tissue graft, the delivery mechanism might be configured to associated secondary materials with a tissue graft from both sides, such as where the jig has a top portion and a bottom portion with associated delivery tissue graft medicator which are inserted into a top and bottom of the tissue graft at the same or different times.

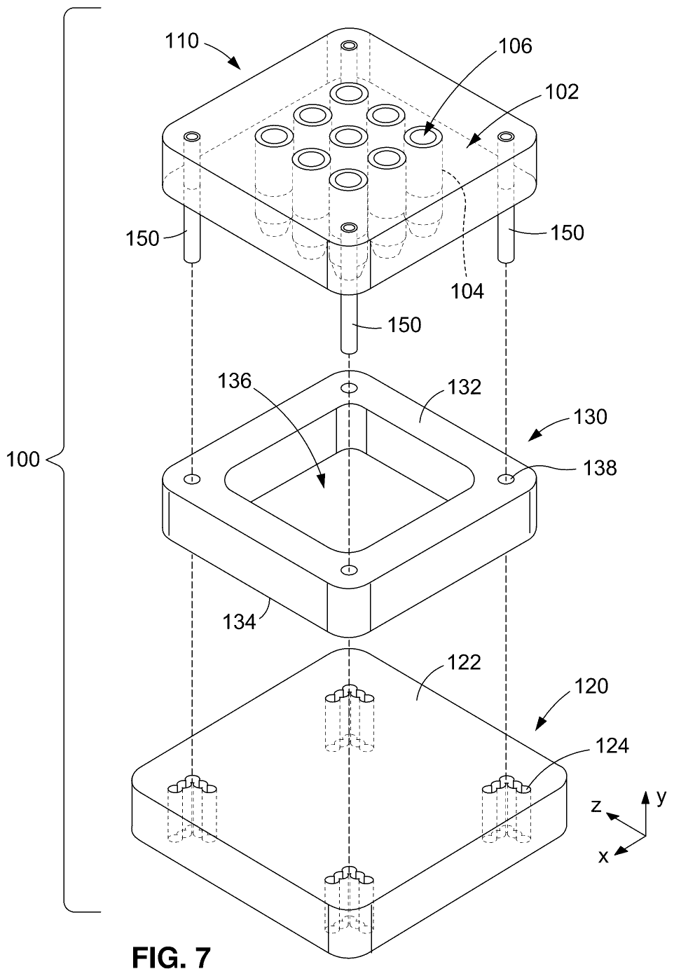

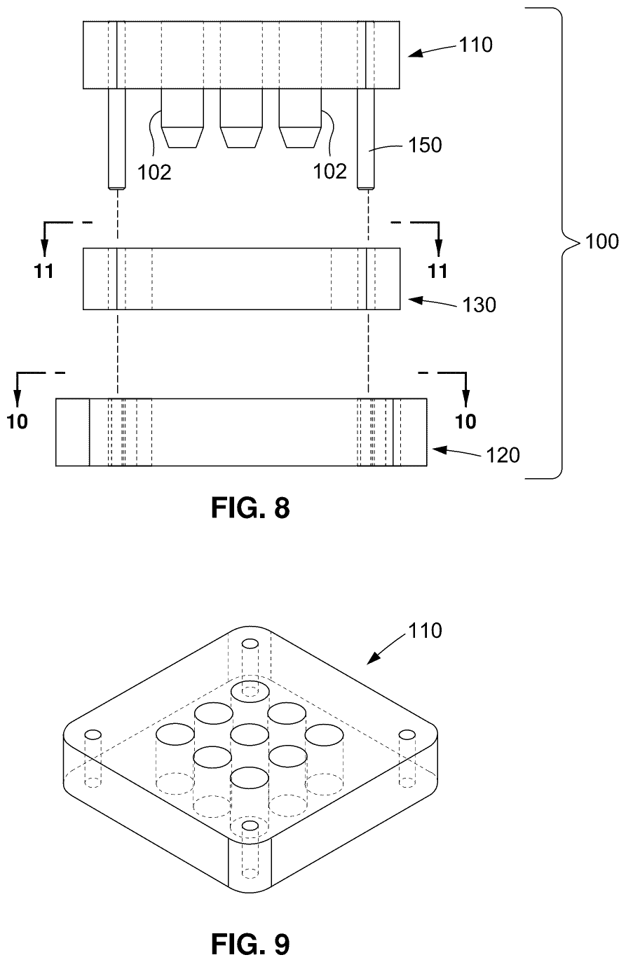

FIGS. 7-11 illustrate another aspect of the present invention. As illustrated, a tissue graft medicator 100 comprises one or more punches 102. As described below, the punches 102 may be used to medicate a tissue graft, such as by cutting the graft or removing material from the graft, and delivering secondary materials (such as medicants, as detailed herein) to the graft.

In one embodiment, the punches 102 comprise a cylindrical wall 104 which defines an internal passage 106. The punch 102 has a proximal end and a distal end, where the internal passage 106 preferably extends inwardly from the proximal end, preferably to the distal end. The proximal end may be connected to a support structure, such as a body 110. In one embodiment, the wall 104 may be tapered at the distal end of the punch 102, so that the distal end defines a cutting edge which is circular in shape.

The number of punches 102 which are associated with the body 110 (including based upon the size of the body 110), as may the spacing between the punches and their orientation relative to one another, may vary. In the embodiment in FIG. 7, the modifier 100 comprise a cross-linear matrix of M rows x N columns of punches 102, where M=N=3. However, the number of punches 102 might be greater, the punches might not be arranged in aligned rows and columns, etc.

In one embodiment of a method, the punches 102 may be moved from a position in which they are retracted from and don't contact a tissue graft, to an inserted position in which they contact a graft. In one embodiment, tissue graft medicator 100 further comprises a platform 120 for supporting a graft to be modified, and may include one or more spacers 130.

The platform 120 preferably defines a graft supporting surface 122. In one embodiment, this surface 122 may comprise a top, planar surface on which a tissue graft may be placed.

The spacer 130 preferably has a top surface 132 and a bottom surface 134 and is designed to be placed between the body 110 and the platform 120. In one embodiment, the spacer 130 defines a central passage 136 from the top surface 132 to the bottom surface 134 through which the punches 102 may extend.

In one embodiment, the tissue graft medicator 100 may include means for guiding the movement of the punches 102. Preferably, the punches 102 are constrained to move linearly perpendicular to the top surface of the platform 120, such as in a “y” direction as illustrated in FIG. 7. In one embodiment, the means for guiding may comprise one or more guide rods 150. The guide rods 150 may be cylindrical bodies (but might have other shapes). In one embodiment, the guide rods 150 extend outwardly from a bottom of the body 110. In the example illustrated, the body 110 as a quadrilateral shape, with four (4) corners, and one guide rod 150 located at each corner. In other embodiments, the guide rods 150 might extend from the body 110 in other locations, such as between the corners. The number of guide rods 150 might also vary.

In one embodiment, the guide rods 150 are each designed to be inserted into a mating passage 124 which extends into the platform 120 from the top surface thereof. Likewise, each spacer 130 preferably has a passage 138 corresponding to each guide rod 150, where the passage 138 extends therethough from the top surface 132 to the bottom surface 134. In use, the guide rods 150 of the body 110 are aligned with mating passages 138 in the one or more spacers 130 (if used) and the platform 120. The body 110 can then be raised and lowered relative to the platform 120, and thus any tissue graft supported thereby. In the embodiment illustrated, movement of the body 110 (and thus the associated punches 102) is constrained by location of the guide rods 150 in the passages of the spacer 130 and platform 120, so that only linear motion is permitted (transverse to a plane containing the top surface 122 of the platform 120). In this regard, in one embodiment, the length of the guide rods 150 is preferably sufficient to ensure that the free ends remain in the passages 124 in the platform 120 as the body 110 is moved up and down.

In one embodiment, means may be provided for shifting the position of the body 110 (and thus the punches 102) or, in other words, to move the body 110 in a plane parallel the plane containing the top surface 122 of the platform 120. In one embodiment, the platform 120 may define a plurality of different sets of passages 124 which correspond to the guide rods 150. The different sets of passages 124 may comprise, for example, a first set of passages and a second set of passages, where the second set of passages is offset in one or more directions (such as an “x” or “z” direction, or combination thereof, as illustrated in FIG. 7).

For example, FIG. 7 illustrates an embodiment in which a plurality of sets of passages 124 are provided. In this embodiment, the passages 124 partially overlap. However, the passages might be separated from one another.

In one embodiment, movement of the body 110, and thus the punches 102, relative to the platform 124 and thus a tissue graft supported thereby, may be done manually. For example, the body 110 may be moved to a position in which the guide rods 124 align with a set of passages 124 in the platform 120. The body 110 may then be moved linearly downward to engage the punches 102 with a tissue graft which is located on the platform 120. The body 110 may also be raised or retracted and moved so that the guide rods 150 align with another set of passages 124 in the platform 120, and the process may be repeated.

In one embodiment, the punches 102 may be used to create partial-depth cuts in a tissue graft. In this configuration, at least one spacer 130 may be used to control the maximum distance that the punches 102 can be moved towards a tissue graft which is located on the top surface 122 of the platform 120. It will be noted that spacers 130 may be provided which have different thicknesses (between the top and bottom surfaces) and/or multiple spacers 130 might be utilized, in order to generate a desired spacing between the distal ends of the punches 102 and the platform 120 for the desired depth of cut into the tissue graft.

In other embodiments, the body 110 and/or the platform 120 might be moved in an autonomous fashion. For example, in one embodiment, the platform 120 might be associated with a motor-driven indexing system, where actuation of one or more motors is configured to cause movement of the platform 120 in the “x” and “z” directions, and where movement of the body 110 transversely to the platform 120 might be via another one or more motors. In such a configuration, the guide rods and passages might be entirely eliminated in favor of these movement/indexing mechanisms, which act to constrain and control the positions of the elements.

It will be appreciated that the shapes of the elements of the tissue graft medicator 100 may vary. For example, while they are all shown as being general square in peripheral shape, they might be circular, rectangular or have other shapes.

While in a preferred embodiment the punches 102 are cylindrical in shape, at least the distal ends of the punches (and in some embodiments, the entire punch) may have other shapes. For example, the distal cutting ends of the punches 102 might be square, triangular, oval or other shapes, including irregular shapes, not limited to wave shaped, C, X or T shaped, etc., thereby resulting in cuts to a tissue graft of such shapes. In some embodiments, different punches 102 of the tissue graft medicator 100 might have different shapes from one another. In other embodiments, the punches 102 might have the same shapes, but might have different sizes (such as different diameters). The shape(s) of the punches 102 may be selected based upon a number of criteria, including: (1) to vary graft exposure to a wound; (2) to alter the absorption/desorption rate of medicant within/from the graft; and (3) to match the characteristics of an anatomic area at which the graft is to be used, such as to insure maximum contact of medicated tissue graft to the anatomic area targeted for treatment. For example, contrasting use of a thin linear punch versus a circular punch, a thin linear punch will create a graft with increased linear contact but decreased vertical contact and will result in a graft which uptakes medicant to the graft faster but also desorb medicant faster from the graft, as compared to use of a circular punch.

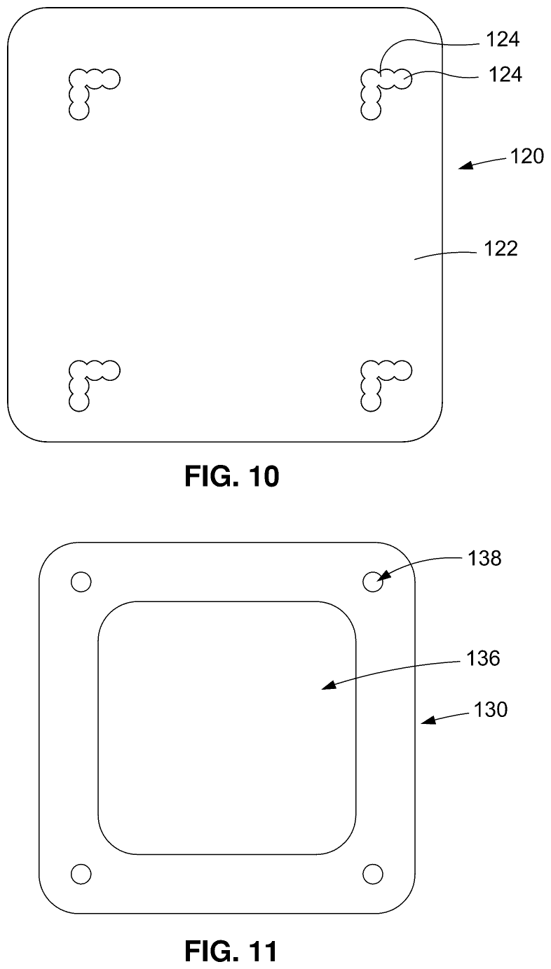

In one embodiment of the invention, the tissue graft medicator 100 may be used in a method in which the graft is located on the platform 120 and the punches 102 are moved into engagement with the graft, such as to create partial depth cuts in the tissue graft (such as a first or top surface thereof, although the tissue graft might then be flipped or turned over to have the second or bottom surface thereof also modified in a similar manner). In one embodiment, the tissue graft is modified in an area corresponding to each of the punches 102 of the tissue graft medicator 100 (e.g. in 9 locations when using the tissue graft medicator 100 which is illustrated in FIG. 7, where there are 9 punches). However, as indicated above, the position of the base 110 and the associated punches 102, may be varied, so that other areas of the tissue graft may be modified. For example, if the base 110 is moved relative to the platform 120 relative to each of the passages 124 in the platform 120 as illustrated in FIGS. 7 and 10, an “L” shaped pattern of overlapping circular cuts is created in the tissue graft.

In a preferred embodiment, the tissue graft modifier 100 is also used to deliver secondary materials to a tissue graft, such as through the passages 106 through the punches. For example, similar to the delivery system 30 described above, the passages 106 in the punches 102 may be connected to one or more sources of secondary materials, such as in the manner described above and illustrated in FIG. 4. In one embodiment, for example, the punches 102 may be lowered until they penetrate the top surface of a tissue graft, thus cutting the graft. The punches 102 might be retained in that position and secondary materials might then be delivered into the tissue graft through one or more of the punches 102.

As indicated above, as one aspect of the invention, the delivery device(s) of the invention may be used to deliver different secondary materials to a tissue graft at different times, such as different secondary materials during different wound healing phases.

In one embodiment of the invention, the secondary materials may comprise oxygen. In one embodiment, this may be done to increase the minimum oxygen tension the tissue graft to >40 mm/hg or >21% O₂ concentration, but not to the point of oxygen toxicity to host cells migrating into the graft.

In one embodiment, the oxygen may be delivered via encapsulated biodegradable spheres which are associated with the tissue graft to achieve an enhanced oxygen environment within the graft. Such spheres may be delivered using a delivery device such as that described above.

In yet another embodiment of the invention, the secondary material delivery device might comprise a pressurized chamber containing secondary materials, such as in gas form. For example, the secondary material might comprise oxygen and the chamber might comprise a hyperbaric chamber. In such a configuration, a tissue graft may be located in a pressurized hyperbaric chamber for variable times, atmospheric pressures, and frequency, in which the tissue graft is exposed to secondary materials (preferably oxygen, in order to achieve an enhanced oxygen environment within the graft). The oxygenated tissue graft may be located in a sealed package, including a pressurized package, such as for delivery and later use.

In certain configurations of the invention, a tissue graft is infused with oxygen in one or more forms (gas, bubble, capsule, etc.), ex-situ, for later association with a host. In other or additional embodiments, oxygen may be provided to the tissue graft in-situ.

In some embodiments, the tissue graft may have one or more layers. As one example, secondary materials might be associated with a tissue graft and then a covering or sealing layer might be applied to one or more surfaces thereof, such as to keep the secondary material entrained in the tissue graft and/or to control the rate at which the secondary material may disassociate itself from the graft. As one example, a tissue graft may be oxygenated and then the top thereof may be sealed, thus forcing the oxygen to migrate from the graft to the sides and/or bottom, into the surrounding host tissue.

In one embodiment, a tissue graft having associated secondary materials may be associated with a host or patient in various manners. As one example, a tissue graft might be placed on top of or over an open wound. In another configuration the tissue graft might be wrapped around host tissue to increase surface contact of the host tissue or organ

In one embodiment, medicants and cellular elements may be associated with a tissue graft for the purpose of treating residual cancer cells on the margins after surgical resection.

One aspect of the invention is the use of a tissue graft having associated secondary materials to allow for transferring/transference of the secondary materials to adjacent host human tissue.

In one embodiment, the secondary materials which are associated with the tissue graft might comprise host tissue. In one configuration, the secondary materials may have the form of cellular elements from an organ donor are associated with a tissue graft (without or with an enriched oxygen environment). For example, the secondary materials might comprise transplanted thyroid tissue cells that secrete thyroxin, pancreatic cells that secrete insulin, or pituitary cells to secrete growth hormone on a long term basis.

In one embodiment, the secondary materials may comprise genetically altered DNA/RNA.

In one embodiment, the secondary material may be selected based on one or more of a healing function of phase, a function of the host tissue, an anatomical area, a disease to be treated, a surgical procedure, or other criteria.

This aspect of the invention has a number of advantages.

The present invention provides for method and apparatus allowing relevant secondary materials to be delivered to tissue graft (and when applied in-situ, to the surrounding tissue). In one configuration, the delivered secondary materials, including by type and amount, may be changed to meet physiologic needs, such as corresponding to the phase of healing, treatment, or transplantation. In one embodiment, one or more secondary materials (not limited to oxygen) may be delivered to a tissue graft ex-situ. After the tissue graft is associated with a host, additional secondary materials may be delivered to the tissue graft in-situ, including differing at different times.

The above description represents various embodiments of the present invention. However, many variations to the method and apparatus are possible without deviating from the scope of the invention. It will be understood that the above described arrangements of apparatus and the method described herein are merely illustrative of applications of the principles of this invention and many other embodiments and modifications may be made without departing from the spirit and scope of the invention as defined in the claims.

What is claimed is:

1. A tissue graft medicator comprising:
a base having a top and a bottom;
a plurality of punches extending from said bottom of said base, said punches having a distal cutting end and a passage extending upwardly from said distal cutting end;
at least two guide rods extending downwardly from said base;
a platform, said platform having a top surface and a plurality of passages extending into said top surface for accepting said at least two guide rods when said base is in two different positions relative to said platform; and
at least one spacer for positioning between said base and said platform, said spacer having a top and a bottom, a main passage therethrough from said top to said bottom through which said plurality of punches may be extended towards said platform, and a passage corresponding to each of said guide rods, through which said guide rods extend into engagement with said platform.

2. The tissue graft medicator in accordance with claim 1, wherein said plurality of punches are cylindrical in shape.

3. The tissue graft medicator in accordance with claim 1, wherein each of said plurality of punches has a proximal end opposite said distal end and said passage extends through said punch from said distal end to said proximal end.

4. The tissue graft medicator in accordance with claim 1, wherein said distal end of each of said plurality of punches is tapered to define a cutting edge.

5. The tissue graft medicator in accordance with claim 1, wherein at least two of said plurality of passages extending into said top surface of said platform intersect one another.

6. The tissue graft medicator in accordance with claim 1, wherein said at least one spacer comprises two or more spacers.

7. The tissue graft medicator in accordance with claim 1, further comprising at least one delivery passage extending into communication with said passages of said plurality of punches for delivering secondary materials to said plurality of punches.

8. A method of medicating a tissue graft, comprising the steps of:
locating a tissue graft having a top surface and a bottom surface on a top of a platform;
moving distal ends of a plurality of punches extending from a base towards said platform and into engagement with said top surface of said tissue graft;
limiting a distance of travel of said plurality of punches by at least one spacer positioned between said base and said platform so that said distal ends of said punches do not pass through said tissue graft from said top surface to said bottom surface; and
associating at least one secondary material with areas of said graft where said punches have engaged said graft.

9. The method in accordance with claim 8, further comprising guiding said punches by sliding one or more guide rods extending from said base through first corresponding passages through said at least one spacer and second corresponding passages extending into said top of said platform.

10. The method in accordance with claim 8, further comprising the step of delivering said at least one secondary material through a passage through each of said plurality of punches while said distal ends thereof are positioned in said tissue graft.

11. The method in accordance with claim 10, wherein said secondary material is selected from the group consisting of: a medicant, an external cellular matrix factor, a cell, an enzyme, and an element or compound.

12. The method in accordance with claim 10, wherein the element comprises oxygen.

13. The method in accordance with claim 8, wherein said tissue graft comprise at least one of cadaveric human tissue, harvested human tissue, amniotic and chorionic tissue.

14. The method in accordance with claim 8, wherein said plurality of punches are generally cylindrical in shape.

15. The method in accordance with claim 8, wherein said distal ends of said plurality of punches are tapered to define a cutting edge.

16. The method in accordance with claim 8, wherein said at least one spacer defines a main passage through said plurality of punches extend towards said platform.
